(19) United States
(12) Patent Application Publication
(10) Pub. No.: US 2021/0346317 A1
(43) Pub. Date: Nov. 11, 2021

(54) TREATMENT OF HEMATOPOIETIC STEM CELL TRANSPLANT PATIENTS

(71) Applicant: PRIOTHERA LIMITED, Dublin (IE)

(72) Inventors: Christoph BUCHER, Basel (CH); Peter GERGELY, Basel (CH); Andreas KATOPODIS, Basel (CH); Philip SMITH, Basel (CH)

(73) Assignee: PRIOTHERA LIMITED, Dublin (IE)

(21) Appl. No.: 17/330,298

(22) Filed: May 25, 2021

Related U.S. Application Data
(63) Continuation of application No. 16/080,949, filed on Aug. 29, 2018.
(60) Provisional application No. 62/305,003, filed on Mar. 8, 2016.

(51) Int. Cl. A61K 31/137; A61K 38/13; A61K 31/255; A61K 31/519; A61K 31/661; A61K 31/675; A61K 31/7076; A61P 41/00

(52) U.S. Cl. CPC A61K 31/137; A61K 38/13; A61K 31/255; A61K 31/519; A61K 2300/00; A61K 31/675; A61K 31/7076; A61P 41/00; A61K 31/661

(57) ABSTRACT

The present invention relates to a method of accelerating stem cell engraftment in a patient in need of hematopoietic stem cell transplantation.

### CROSS-REFERENCE TO RELATED APPLICATIONS

This application is a continuation of U.S. patent application Ser. No. 16/080,949, filed Aug. 29, 2018, which is a U.S. National Stage Application filed under 35 U.S.C. § 371 of International Application No. PCT/IB2017/051291, filed Mar. 6, 2017, which claims priority to and the benefit of U.S. Provisional Application No. 62/305,003, filed Mar. 8, 2016, the contents of each of which are hereby incorporated by reference in their entireties.

The present invention relates to a method of treating patients who undergo hematopoietic stem cell transplantation (HSCT) with peripheral blood mobilized stem cells or blood marrow transplantation for hematological malignancies and for whom a faster engraftment is beneficial.

### BACKGROUND

Hematopoietic stem cell transplantation (HSCT) is the transplantation of multipotent hematopoietic stem cells, usually derived from bone marrow, peripheral blood, or umbilical cord blood. It may be autologous (the patient's own stem cells are used) or allogeneic (the stem cells come from a donor). It is a medical procedure in the field of hematology, and a potentially curative approach for a variety of malignant and nonmalignant hematopoietic diseases, such as e.g. cancers of the blood or bone marrow, e.g. lymphoma or leukemia. The cells that are transplanted can be unmodified or genetically engineered (e.g. Lentiviral, CRISPR). When HSCT is performed in patients with malignant disorders, preparative or conditioning regimens are administered before the transplantation in order to destroy or restrict the recipient's immune system (e.g. non-myeloablative, partial or full myeloablation) to prevent graft rejection and reduce the tumor burden. Such conditioning treatments may consist of performing radiation and/or chemotherapy.

Delivering (autologous or allogeneic) stem cells with a minimal toxicity is an unmet medical need because a substantial fraction of post transplant mortality and morbidity is related to conditioning toxicity. However, delivering stem cells after non-myeloablative conditioning has a risk of graft failure which prevents the successful outcome of a transplant procedure altogether.

In nonmalignant diseases the anti-neoplastic effect of conditioning is not needed. Therefore there is a need to use a less myeloablative or less immunosuppressive conditioning treatment and/or limit the possible consequences thereof, e.g. side-effects, that are possibly associated with such conditioning regimens. Ideally, radiation and chemotherapy could be replaced by novel, non-toxic approaches entirely.

In some circumstances it would be advantageous to use a more reduced dose of hematopoietic cells for the transplantation than usually necessary for obtaining engraftment. But the use of less HSC would require a more intense conditioning, which has higher toxicity and provides a slower reconstitution.

Once hematopoietic stem cells have been transplanted the speed of engraftment plays an important and direct role for patients who have undergone blood marrow transplantation myeloablative conditioning, because every additional day of cytopenia (i.e. reduction in the number of blood cells), especially neutropenia (lower level of neutrophils) and thrombopenia (lower level of platelets), is usually associated with additional morbidity and mortality. This morbidity and mortality are caused by infections, bleeding and need for transfusion support both of red blood cells and platelets.

Improving the engraftment, e.g. accelerating it, may also permit the patient to leave the hospital earlier and/or recover quicker from the surgery.

Therefore there is a high unmet medical need to have a pharmaceutically effective drug which will make HSCT safer for both donor and recipient and may allow enlarging the patient populations that could benefit from HSCT. There is also a need to perform HSCT in patients who do not tolerate classical conditioning, e.g because the conditioning treatment would have severe or even life-threatening consequences on such patients, e.g. patients with metabolic diseases, elderly, juvenile patients or patients with comorbid conditions. It can also be patients for which it would not be justified to take the risks possibly associated with chemotherapy or radiotherapy of classical conditioning regimens in view of the nature of the disease affecting these patients, e.g. patients with non-malignant indications.

Unexpectedly, it was found that a compound according to formula (I), or a pharmaceutically acceptable salt thereof, and in particular KRP203, significantly increases the speed of HSC engraftment. Therefore the use of said compound permits to minimize the days of cytopenia to the shortest possible period and/or to use a lower cell dose or reduced conditioning. It also permits to render the HSCT easier and safer for the donor, and thus to identify donors more easily, to perform more transplantation and a larger panel of patients in need thereof.

### SUMMARY OF THE INVENTION

In a first embodiment the present invention relates to a method of improving, e.g. facilitating, e.g. accelerating hematopoietic stem cell engraftment, e.g. neutrophil cell, in a patient undergoing HSCT (hematopoietic stem cell transplantation), which method comprises administering an effective amount of a compound of formula (I) or a pharmaceutically acceptable salt thereof to said patient,

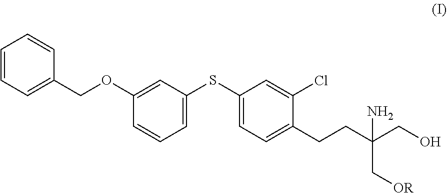

wherein R is H or P(O)₃H₂.

In another embodiment the invention relates to a compound of formula (I) or a pharmaceutically acceptable salt thereof for use of accelerating engraftment of hematopoietic stem cells in a patient who received a hematopoietic stem cell transplantation (HSCT) from a donor.

As used herein a compound of formula (I) or a pharmaceutically acceptable salt thereof, especially a hydrochloride salt, is selected from

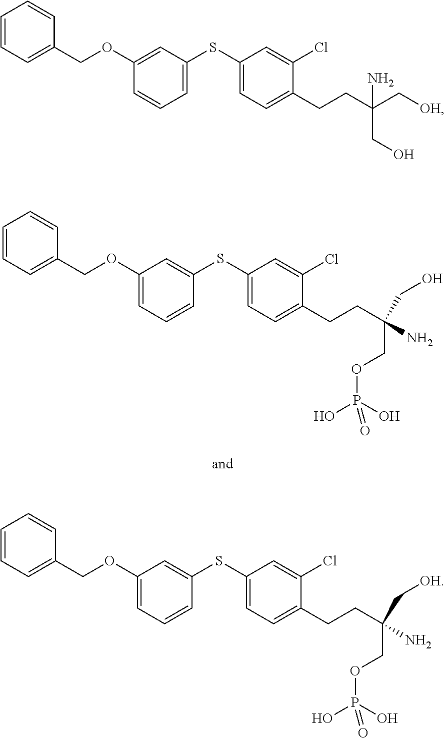 [SMILES: NC(CO)(CO)CCc1ccc(Sc2cccc(OCc3ccccc3)c2)cc1Cl ; N[C@](CO)(CCc1ccc(Sc2cccc(OCc3ccccc3)c2)cc1Cl)COP(=O)(O)O ; N[C@@](CO)(CCc1ccc(Sc2cccc(OCc3ccccc3)c2)cc1Cl)COP(=O)(O)O]

As used herein KRP203 refers to

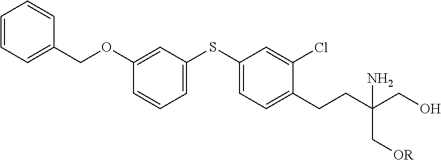

or a pharmaceutically acceptable salt thereof.

The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

As used herein a daily dosage refers to the daily amount of the pure active ingredient, i.e. a compound of formula (I) or a pharmaceutically acceptable salt thereof, e.g. 2-amino-2-[4-(3-benzyloxyphenylthio)-2-chloro-phenyl]ethyl-propane-1,3-diol hydrochloride.

As used herein the term “conditioning” or “conditioned” in the context of a patient pretreatment in need of HSCT typically means destroying substantially the bone marrow and immune system by a suitable procedure such as e.g. chemotherapy and/or radiation therapy.

Conditioning regimens have been classified as high-dose (myeloablative), reduced-intensity, and nonmyeloablative, following the Reduced-Intensity Conditioning Regimen Workshop, convened by the Center for International Blood and Marrow Transplant Research (CIBMTR) during the Bone Marrow Transplantation Tandem Meeting in 2006.

The method according to the present invention, e.g. improving or accelerating engraftment, also permits to use a less myeloablative or less immunosuppressive conditioning treatment and/or limit the possible consequences, e.g. side effects, that are possibly associated with such conditioning regimens. In some circumstances, the methods of the present invention may even permit to perform HSCT without any conditioning.

The method of the present invention may also permit to use a reduced dose of hematopoietic cells for the transplantation than would otherwise be necessary for obtaining engraftment.

According to the invention, the method of performing hematopoietic stem cells transplantation may permit to use less HSC than otherwise require, e.g. without requiring a more intense conditioning.

Thus the present invention will make HSCT safer for both donor and recipient and may allow enlarging the patient populations that could benefit from HSCT. The present invention permits to perform HSCT in patients who do not tolerate a classical conditioning, e.g because the conditioning treatment would have severe or even life-threatening consequences on them, e.g. patients with metabolic diseases, elderly, juvenile patients or patients with comorbid conditions. The present invention also permits to perform HSCT in patients where taking the risks that can be associated with the chemotherapy or radiotherapy of the classical conditioning regimens would not be justified in view of the nature of their disease, e.g. patients with non-malignant indications. Because of the present invention, HSCT treatment can be utilized in a larger patients population, in particular in patients who could not have had access to such a technology because of their overall physical condition, age and/or the specific disease they are affected by.

As illustrative the following techniques can be used:

Non-myeloablative conditioning (NMA e.g. Mini-Seattle Conditioning), e.g. fludarabin or another chemotherapeutic agent typically at 30 mg/m2/day for three days followed by total body irradiation (TBI) typically at 1×200 cGy/day for one day;

Reduced intensity conditioning (RIC; typically FluBu);

Myeloablative conditioning, g (MAC; typically CyTBI of BuCy);

or

e.g. high dose chemotherapy and total body irradiation (TBI) is typically performed according to national guidelines adapted to institutional practices, and includes the administration of fludarabin, busulphan.

The following dosing regimens are given as examples:

1) Fludarabin at 25 mg/m2/day i.v. ×3 days (for approximately 2-3 days) for a total dose of 75 mg/m2,

2) Busulphan at 0.8 mg/kg/6 h (for approximately 2 to 4 days),

3) Cyclophosphamide at 60 mg/kg/day i.v. ×2 days (approximately for 2 days) for a total dose of 120 mg/kg. To reduce the risk of CYC-induced hemorrhagic cystitis, patients will also receive high volume fluid flushes and mesna.

4) TBI will occur from approximately days 8 to 10 (days −8 and −1 relative to HSCT).

The recommended TBI dose is 200 cGy given twice daily for three days for a total dose of 1200 cGy.

### BRIEF DESCRIPTION OF THE DRAWINGS

FIG. 1 shows the probability of neutrophil engraftment of the control group versus the group of patients treated with KRP203.

### DETAILED DESCRIPTION OF THE INVENTION

Embodiment 1 describes a method of accelerating engraftment of hematopoietic stem cells in a patient who received hematopoietic stem cells via transplantation from a donor, which method comprises administering to the patient an effective amount of a compound of formula (I) or a pharmaceutically acceptable salt thereof, e.g. KRP203 or a pharmaceutically acceptable salt thereof, e.g. KRP203 hydrochloride salt.

As used herein, accelerating engraftment refers to obtaining, e.g. detecting, engraftment after the cell infusions, e.g. detecting the first day of engraftment, in a shorter period of time than without administering an effective amount of a compound of formula (I) as herein defined.

The first day of engraftment is usually defined as the 1^{st} day after a period of 3 consecutive days where the amount of neutrophils present in the blood, as determined e.g. by routine hematograph, is ≥500/μl.

Duration of engraftment also depends on the conditioning treatment that occurred or not before the transplantation. Without KRP203 therapy that period is usually around 15 to 16 days after cell infusion in case of full conditioning (myeloablative).

Embodiment 2 describes a compound of formula (I) or a pharmaceutically acceptable salt thereof, e.g. KRP203 or a pharmaceutically acceptable salt thereof, for use in accelerating engraftment of hematopoietic stem cells in a patient who received said hematopoietic stem cells via a transplantation procedure from a donor.

Embodiment 3 describes a method or a compound according to any of the preceding embodiments, e.g. KRP203 or or a pharmaceutically acceptable salt thereof, wherein said patient is first subjected to conditioning, e.g. myeloablative, reduced-intensity or nonmyeloablative, e.g. myeloablative conditioning, e.g destroying substantially all the bone marrow of the patient.

Embodiment 4 describes a method or a compound in accordance to embodiment 3 wherein said conditioning is a high dose chemotherapy comprising one or more agents selected from fludarabin, busulphan, methotrexate, cyclosporin A and cyclophosphamide.

Embodiment 5 describes a method or a compound in accordance to embodiment 3 or 4, wherein said conditioning is a total body irradiation (TBI) according to national guidelines.

Embodiment 6 describes a method or a compound in accordance to any of the embodiments 3 to 5, wherein hematopoietic stem cell transplantation (HSCT) is carried out following to conditioning, e.g. immediately after conditioning, or 0-1 day after conditioning, or 1-8 days, or 1-10 days after conditioning.

Embodiment 7 describes a method or a compound in accordance to any of the embodiments 3 to 6, wherein treatment of the patient with a compound of formula (I) is commenced 5 days before conditioning, in particular 3 days before conditioning, e.g. 1 day before conditioning.

Embodiment 8 describes a method or a compound according to any of the preceding embodiments, e.g. KRP203 or a pharmaceutically acceptable salt thereof, e.g KRP203 hydrochloride, wherein said patient is subject to non-myeloablative conditioning or is not subjected to a conditioning regimen prior to the cell infusion, e.g. said patient is affected by a non-malignant condition, a non-hematological condition, is a juvenile, is an elderly patient, e.g. over 55 years old, or does suffer from a comorbid condition.

Embodiment 9 describes a method for performing hematopoietic stem cell transplantation (HSCT) in a patient in need thereof, wherein a compound of formula (I) or a pharmaceutically acceptable salt thereof, e.g. KRP203 or a pharmaceutically acceptable salt thereof, e.g. KRP203 hydrochloride salt, is administered to the patient and wherein said patient does not undergo a conditioning regimen before the transplantation or does undergo a non-myeloablative conditioning, and wherein optionally the patient is affected by a non-hematological condition.

Embodiment 10 describes a method for performing hematopoietic stem cells transplantation in a patient in need thereof wherein the patient receives the cells from a donor, which method comprises administering to said patient a number of donor cells up to 50% lower than otherwise needed without administering the compound of formula (I), e.g. up to 40% lower than otherwise needed without administering the compound of formula (I), e.g. up to 30% lower than otherwise needed without administering the compound of formula (I), e.g. up to 20% lower than otherwise needed without administering the compound of formula (I), e.g. up to 10% lower than otherwise needed without administering the compound of formula (I). Optionally the patient is subjected to a conditioning regimen before the transplantation, e.g. a non-myeloablative regimen.

Embodiment 11 describes a method for performing hematopoietic stem cells transplantation in a patient in need thereof wherein the patient receives the cells from a donor, which method comprises administering to said patient a number of CD34 cells comprised between about 1×10E6 cells/kg recipient to about 9×10E6 cells/kg recipient, e.g. about 1×10E6 cells/kg recipient to about 8×10E6 cells/kg recipient, e.g. about 1×10E6 cells/kg recipient to about 7×10E6 cells/kg recipient, e.g. about 1×10E6 cells/kg recipient to about 6×10E6 cells/kg recipient, e.g. about 1×10E6 cells/kg recipient to about 5×10E6 cells/kg recipient, e.g. about 1×10E6 cells/kg recipient to about 4×10E6 cells/kg recipient. Optionally the patient is subjected to a conditioning regimen before the transplantation, e.g. a non-myeloablative regimen.

Embodiment 12 describes a method for performing hematopoietic stem cells transplantation in a patient in need thereof wherein the patient receives a dose of cells from the donor of about 1×10E6 cells/kg recipient, or about 2×10E6 cells/kg recipient, or about 2×10E6 cells/kg recipient, or about 2×10E6 cells/kg recipient or about 5×10E6 cells/kg recipient. Optionally the patient is subjected to a conditioning regimen before the transplantation, e.g. a non-myeloablative regimen. Optionally the patient is subjected to a conditioning regimen before the transplantation, e.g. a non-myeloablative regimen.

Embodiment 13 describes a method in accordance to any of the preceding embodiments, wherein the period until the first day of engraftment is obtained within at least one day up to one week, e.g. at least one day, at least 2 days, at least 3 days, at least 4 days, at least 6 days.

Embodiment 14 describes a method in accordance to any of the embodiments 1 to 13, wherein the engraftment of hematopoietic stem cells in a patient in need thereof is obtained in about 12 to 14 days, e.g. about 12 to 13 days, after the stem cell infusion, in particular wherein a conditioning treatment was performed before the cell infusion, e.g. a myeloablative or non myeloablative conditioning.

Embodiment 15 describes a method in accordance to any of the preceding embodiments, e.g KRP203 or a pharmaceutically acceptable salt thereof, e.g KRP203 hydrochloride, wherein said patient is selected from a patient suffering from a malignant disease, a non-malignant indication, a metabolic disease, and a comorbid condition and an autoimmune disease, e.g. as herein defined, e.g. aplastic anemia, hemoglobinopathy, thalassemia, Sickle disease, Hurler's disease.

Embodiment 16 describes a method for performing hematopoietic stem cell transplantation (HPSC) in a patient suffering from a metabolic disease, e.g. hemoglobinopathy, aplastic anemia, thalassemia, Sickle disease, Hurler's disease, and/or an elderly patient, e.g. over 55 years old, or a juvenile patient, wherein said method comprises administering to said patient a compound of formula (I) or a pharmaceutically acceptable salt thereof, e.g. KRP203 or a pharmaceutically acceptable salt thereof, e.g. KRP203 hydrochloride. The method may or not comprise a conditioning treatment before cell transplantation, e.g does comprise a non-myeloablative conditioning regimen.

Embodiment 17 describes a method for treating a metabolic disease, e.g. hemoglobinopathy, aplastic anemia, thalassemia, Sickle disease, Hurler's disease, in a patient undergoing hematopoietic stem cell transplantation (HSCT), which method comprises:

(i) Administering to the patient an effective amount of a compound of formula (I) or a pharmaceutically acceptable salt thereof, e.g. KRP203 or a pharmaceutically acceptable salt thereof, e.g. KRP203 hydrochloride;

(ii) Conditioning said patient prior the transplantation, e.g. performing a non-myeloablative conditioning; and

(iii) Transplanting hematopoietic stem cells from a donor to said patient.

Embodiment 18 describes a method for performing hematopoietic stem cell transplantation (HSCT) in a patient in need thereof, e.g. a juvenile, an elderly (e.g. over 55 years old), which method comprises:

(i) Administering to the patient an effective amount of a compound of formula (I) or a pharmaceutically acceptable salt thereof, e.g. KRP203 or a pharmaceutically acceptable salt thereof, e.g. KRP203 hydrochloride;

(ii) Conditioning said patient prior the transplantation, e.g. performing a non-myeloablative conditioning; and

(iii) Transplanting hematopoietic stem cells from a donor to said patient.

Embodiment 19 described a compound according to any of the preceding embodiments, e.g. KRP203 or or a pharmaceutically acceptable salt thereof, e.g KRP203 hydrochloride, for use in a method according to any one of the embodiments 9 to 18.

Embodiment 20 describes a method or a compound in accordance to any of the preceding embodiments, wherein said compound, e.g KRP203 or a pharmaceutically acceptable salt thereof, e.g KRP203 hydrochloride, is administered at a daily dose of 1, 2, 3, 4 or 5 mg (per patient), e.g. daily dose of 1 mg or 2 mg or 3 mg. The daily dose of the compound of formula (I), e.g. KRP203 or or a pharmaceutically acceptable salt thereof, e.g. hydrochloride salt, may be administered once a day, or several times a day, e.g. once a day. The compound of formula (I), e.g. KRP203 or or a pharmaceutically acceptable salt thereof, e.g. hydrochloride salt, may also be administered at longer intervals, e.g. once a week, or every 4-5 days. According to the invention, the donor may the patient who receives his or her own stem cells (autologous transplantation), or another person (allogeneic transplantation). The source of the stem cell can be umbilical cord, haploidentical.

Embodiment 21 describes a method or a compound in accordance to any of the preceding embodiments, wherein said compound is administered at a daily dose of 1, 2 or 3 mg (per patient). The daily dose of the compound of formula (I), e.g. KRP203 or a pharmaceutically acceptable salt thereof, may be administered once a day, or several times a day, e.g. once a day.

Embodiment 22 refers to a compound of formula (I), e.g. KRP203 or a pharmaceutically acceptable salt thereof, for use in performing HSCT in a patient in need thereof, comprising administering a daily dose of about 1 mg to 3 mg, e.g about 1 mg, e.g. about 2 mg, e.g. about 3 mg.

Embodiment 23 refers to a method for performing HSCT in a patient in need thereof, comprising administering to a patient in need thereof compound of formula (I), e.g. KRP203 or a pharmaceutically acceptable salt thereof at a daily dose of about 1 mg to 3 mg, e.g about 1 mg, e.g. about 2 mg, e.g. about 3 mg.

Embodiment 24 refers to a compound of formula (I), e.g. KRP203 or a pharmaceutically acceptable salt thereof, for use in treating a non-malignant condition or a non-hematological condition comprising administering a daily dose of about 1 mg to 3 mg, e.g about 1 mg, e.g. about 2 mg, e.g. about 3 mg, and comprising performing HSCT in the patient. Optionally, the patient is a selected from a juvenile, an elderly patient and a patient who suffers from a comorbid condition, e.g. a patient over 55 years old.

Embodiment 25 refers to a method for treating a non-malignant condition or a non-hematological condition in a patient in need thereof, comprising the steps of i) performing HSCT in the patient and ii) administering to a patient in need thereof compound of formula (I), e.g. KRP203 or a pharmaceutically acceptable salt thereof at a daily dose of about 1 mg to 3 mg, e.g about 1 mg, e.g. about 2 mg, e.g. about 3 mg. Optionally, the patient is a selected from a juvenile, an elderly patient and a patient who suffers from a comorbid condition, e.g. a patient over 55 years old.

The patient (receptor) may be affected by a metabolic disease, a malignant disease such as a blood cancer, e.g. by one disease selected from leukemia, multiple myeloma, lymphoma, e.g. acute lymphoblastic leukemia, acute myeloblastic leukemia, chronic lymphoid leukemia, myelodysplastic syndrome, non-Hodgkin lymphoma. In another embodiment, the patient is affected by a non-malignant disease, e.g. aplastic anemia; a metabolic disease or disorder, e.g. hemoglobinopathy, thalassemia, Sickle disease or Hurler's disease. The patient may have a comorbid condition, e.g. a heart disease, AIDS or cancer. The patient may be affected by bone marrow transplantation combined with solid organ transplantation for tolerance induction. The patient may suffer from autoimmune diseases such as e.g. type I diabetes, multiple sclerosis, scleroderma.

Embodiment 26 describes a method for treating or preventing one disease amongst the diseases mentioned above, e.g. a malignant disease, a non-malignant disease, a comorbid condition or an autoimmune disease, wherein said method comprises:

(i) Administering to the patient an effective amount of a compound of formula (I) or a pharmaceutically acceptable salt thereof;

(ii) Transplanting hematopoietic stem cells from a donor to said patient; and optionally

(iii) Performing a conditioning to said patient prior the transplantation, e.g. performing a non-myeloablative conditioning. The compound of formula (I) or a pharmaceutically acceptable salt thereof, e.g. KRP203 or a pharmaceutically acceptable salt thereof, e.g. KRP203 hydrochloride, can be administered at a daily dose of about 0.5 mg to 5 mg, e.g. about 1 mg to about 3 mg, e.g. about 0.5 mg, e.g. about 1 mg, e.g. about 3 mg,

Embodiment 27 refers to a compound of formula (I) or a pharmaceutically acceptable salt thereof, e.g. KRP203 or a pharmaceutically acceptable salt thereof, e.g. KRP203 hydrochloride salt for use in a method of accelerating engraftment of hematopoietic stem cells in a patient who received hematopoietic stem cells via transplantation from a donor.

According to the invention, the patient may be medically infirm, elderly e.g. over 55 years old, e.g. over 60 years old), juvenile, e.g. children with severe combined immunodeficiency.

The compound of formula (I), e.g. KRP203 or or a pharmaceutically acceptable salt thereof, e.g. KRP203 hydrochloride, may be administered after the transplantation, e.g. during up to 150 days post-transplantation, e.g. up to 120 days post-transplantation, e.g. up to 100 days post-transplant, e.g. during 3 months, e.g. during one months, e.g. during one week after transplantation. For example, the compound of formula (I), e.g. KRP203 or or a pharmaceutically acceptable salt thereof, e.g. KRP203 hydrochloride, may be administered during about 120 days after the transplantation, e.g. about 110 days after the transplantation, e.g about 100 days after the transplantation, e.g about 90 days after the transplantation.

According to the invention, administration of the compound of formula (I), e.g. KRP203 or or a pharmaceutically acceptable salt thereof, e.g. KRP203 hydrochloride, can start before transplantation, e.g 1 to 3 days before the transplantation (e.g. one day, or two days before the transplantation), or at the day of transplantation. The duration of administering the compound of formula (I), e.g. KRP203 or or a pharmaceutically acceptable salt thereof, e.g. KRP203 hydrochloride, can be of about 120 days, e.g. about 110 days, e.g about 100 days after the transplantation, e.g about 90 days.

According to the invention, the donor may the patient who receives his or her own stem cells (autologous transplantation), or another person (allogeneic transplantation).The patient (receptor) may be affected by one disease selected from leukemia, multiple myeloma, lymphoma, e.g. acute lymphoblastic leukemia, acute myeloblastic leukemia, chronic lymphoid leukemia, myelodysplastic syndrome, non-Hodgkin lymphoma.

Treatment Procedure

Compound for accelerating engraftment of hematopoietic stem cells:

The compound of formula (I), in particular KRP203, especially compositions comprising 0.5; 1; 2; 3; 4 or 5 mg of 2-amino-2-[4-(3-benzyloxyphenylthio)-2-chlorophenyl]ethyl-propane-1,3-diol or a pharmaceutically acceptable salt thereof are provided. Especially preferred are capsules or tablets comprising 1 or 2 mg 2-amino-2-[4-(3-benzyloxyphenylthio)-2-chloro-phenyl]ethyl-propane-1,3-diol, e.g. as hydrochloride.

The treatment may comprise (representative schedule):

A: A screening period (Days −50 to −2), Baseline (Day −1),

B: Drug treatment period from Day 1 to Day 111 and a follow-up period up to 365 days (from transplant), wherein the drug is a compound of formula (I) or a pharmaceutically acceptable salt thereof, e.g. 2-amino-2-[4-(3-benzyloxyphenylthio)-2-chloro-phenyl]ethyl-propane-1,3-diol, e.g. as hydrochloride,

C: Myeloablative or Mini Seattle Conditioning will be performed between Day 2 and Day 10 as per as described herein,

D: Transplantation (infusion of stem cells), i.e. HSCT will be performed on Day 11.

Standard activities, in addition to the investigative treatment may include standard GVHD prophylaxis, pre and post transplant supportive care and follow-up assessments according to the institutional practices.

Hematopoietic Stem Cells (HSC)

Peripheral mobilized stem cells will be used according to institutional practices. Suitable stem cell source must be available according to the graft selection algorithm as defined by JACIE* adapted to institutional standards using T-cell replete peripheral stem cells as a graft source. (*JACIE: The Joint Accreditation Committee Europe comprising the International Society for Cellular Therapy & European Group for Blood and Marrow Transplantation). In addition, the donor must be 9/10 or 10/10 matched with the recipient using molecular HLA matching techniques.

### EXAMPLES

### Example 1

### Mouse Model of Inherited Metabolic Disease (IMD)

Recipient C57BL/6 or IDUAKI (B6.129S-Iduatm1.1Kmke/J) mice were conditioned with a mild non-myeloablative regimen. A limited number of 1×10E3 HSC were transplanted from congenic mice. Groups were either untreated or treated with KRP for 15 days. More mice from the KRP203 treated group engrafted on day 56 and the engrafted mice had more donor cells than the untreated group. The conclusion is that in non-myeloablative conditions under limited HSC conditions, KRP treatment in the peri-transplant period augments engraftment.

### Example 2

Description of Experiment:

Day 1=drug treatment commences (e.g. 2 mg KRP203/per day)

Day 2 and Day 10: Conditioning was performed as described above (e.g. Mini Seattle or RIC)

Day 11: transplantation of stem cells from an allogeneic donor is being carried out

Day 11: pursuant to the foregoing transplantation procedure, the neutrophil count was determined and was 33% above baseline in the KRP203 group; and 11% above baseline for the control group

Day 15: neutrophil count was 100% above baseline for KRP203 group and 48% above baseline for control group.

To monitor for engraftment of donor hematopoietic stem cells in recipients, the neutrophil count is measured usually daily. The commonly accepted definition of “day of engraftment” (take) is the first of three consecutive days with a neutrophil count higher than 500 cells per microliter. A neutrophil count of higher than 500 cells per microliter is reached in the below experiment, when the probability of engraftment is 1.0 (100%) versus baseline or higher. The donor origin of these cells is normally confirmed by genomic analyses on day 30.

Results

TABLE 1

| | Probability of neutrophil | Probability of neutrophil |
|---|---|---|
| | engraftment | engraftment |
| Day | Control | KRP203 |
| 0 | 0.00 | 0.00 |
| 10 | 0.01 | 0.00 |
| 11 | 0.11 | 0.33 |
| 12 | 0.14 | 0.44 |
| 13 | 0.36 | 0.67 |
| 14 | 0.40 | 0.89 |
| 15 | 0.48 | 1.00 |
| 16 | 0.58 | 1.00 |
| 17 | 0.68 | 1.00 |
| 18 | 0.81 | 1.00 |
| 19 | 0.85 | 1.00 |
| 20 | 0.88 | 1.00 |
| 21 | 0.90 | 1.00 |
| 22 | 0.94 | 1.00 |
| 23 | 0.99 | 1.00 |

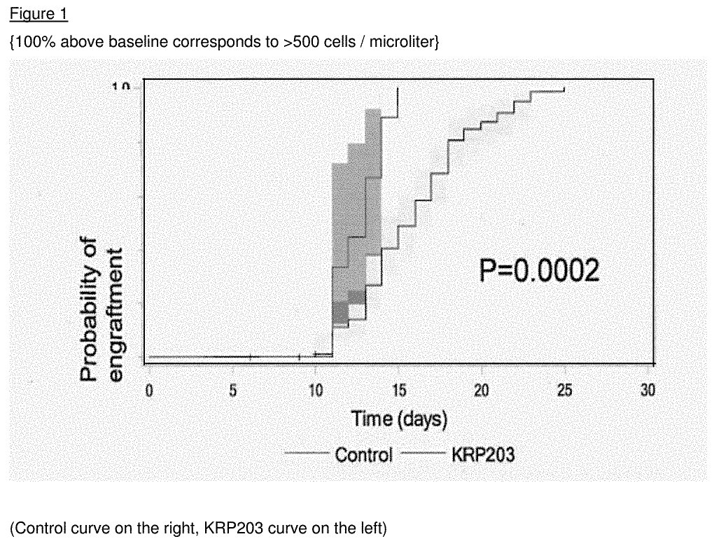

FIG. 1 and Table 1 show the probability of neutrophil engraftment of the control group versus the group of patients treated with KRP203. As it can be seen, the group treated with KRP203 engrafted much faster than the control group.

1.-10. (canceled)

11. A method for treating a metabolic disease in a patient, which method comprises:
(i) administering to the patient an effective amount of a compound of formula (I): 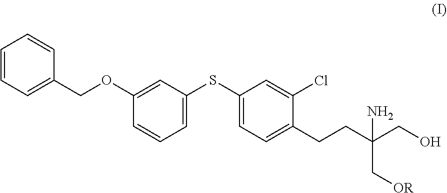
or a pharmaceutically acceptable salt thereof,
wherein R is H or P(O)₃H₂, and,
(ii) transplanting hematopoietic stem cells from a donor to said patient.

12. The method of claim 11 wherein said metabolic disease is selected from the group consisting of Sickle disease, hemoglobinopathy, aplastic anemia, thalassemia, and Hurler's disease.

13. The method of claim 11, wherein said compound is KRP203 or a pharmaceutically acceptable salt thereof.

14. The method of claim 13, wherein the pharmaceutically acceptable salt thereof is KRP203 hydrochloride.

15. The method of claim 11, wherein said compound or a pharmaceutically acceptable salt thereof is selected from:

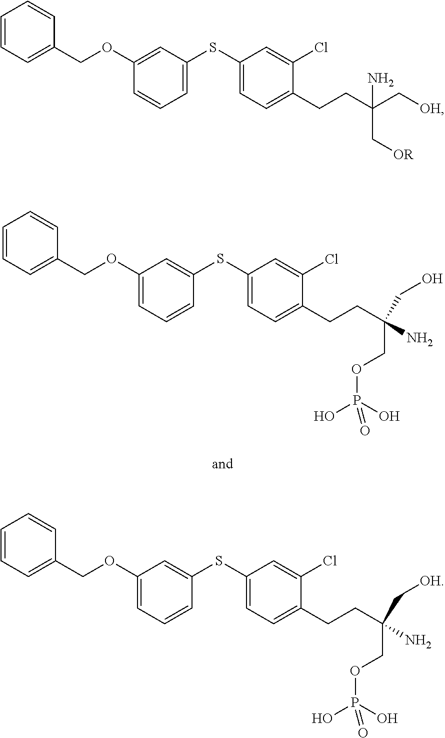

16. The method of claim 11, wherein said compound is administered at a daily dose of 1, 2, 3, 4 or 5 mg (per patient).

17. The method of claim 11, wherein said compound is administered once a day, or several times a day, or once a week, or every 4-5 days.

18. The method of claim 11, wherein said compound is administered after the transplantation.

19. The method of claim 11, wherein said patient is first subjected to conditioning before cell transplantation.

20. The method of claim 19, wherein said conditioning is a non-myeloablative conditioning.

21. The method of claim 19, wherein said conditioning is a chemotherapy comprising one or more agents selected from fludarabin, busulphan, methotrexate, cyclosporin A, and cyclophosphamide followed by total body irradiation.

22. The method of claim 19, wherein said chemotherapy administered at 30 mg/m2/day for three days and said total body irradiation is conducted at 1×200 cGy/day for one day.

23. The method of claim 11, wherein the patient is a juvenile or elderly patient.

24. The method of claim 11, wherein the donor is the patient.

25. The method of claim 11, wherein the donor is not the patient.

26. The method of claim 11, wherein said compound is administered before transplantation.

27. The method of claim 11, wherein said compound is administered 1 to 3 days before the transplantation or at the day of transplantation.

28. The method of claim 11, wherein said compound is administered for about 90 days, after the transplantation.

29. A method of accelerating engraftment of hematopoietic stem cells in a patient who received said hematopoietic stem cells via a transplantation procedure from a donor, said method comprising:
administering to the patient in need thereof an effective amount of a compound of formula (I) or a pharmaceutically acceptable salt thereof, 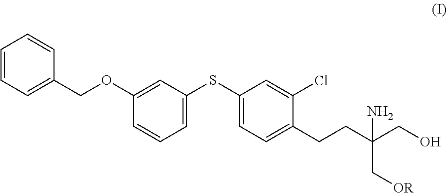
wherein R is H or P(O)₃H₂, and
wherein said patient is selected from a patient suffering from: a non-malignant indication, a metabolic disease, and a comorbid condition and an autoimmune disease.

30. The method of claim 29, wherein said patient is selected from a patient suffering from metabolic disease selected from the group consisting of: hemoglobinopathy, aplastic anemia, thalassemia, Sickle disease and Hurler's disease.

31. The method of claim 29, wherein said patient is an elderly or juvenile patient.

32. The method of claims 29, wherein said patient is first subjected to conditioning before cell transplantation.

33. The method of claim 29, wherein said conditioning is a non-myeloablative conditioning regimen.
